(19) United States
(12) Patent Application Publication
(10) Pub. No.: US 2021/0308440 A1
(43) Pub. Date: Oct. 7, 2021

(54) DEVICE COMPRISING MICRONEEDLES FOR SKIN-COLORING

(71) Applicant: L'OREAL, Paris (FR)

(72) Inventors: Dominique BORDEAUX, Chevilly La Rue (FR); Gaurav AGARWAL, Takatsu ku (FR); Philippe BARBARAT, Chevilly La Rue (FR)

(21) Appl. No.: 17/269,111

(22) PCT Filed: Sep. 24, 2018
(86) PCT No.: PCT/EP2018/075793

§ 371 Date: Feb. 17, 2021

(51) Int. Cl. A61M 37/00

(52) U.S. Cl. CPC A61M 37/0015; A61M 37/0084; A61M 37/0092; A61M 2037/0046; A61M 2037/0061; A61M 2037/003

(57) ABSTRACT

A device for delivering to the skin, scalp, or lips a skin-coloring cosmetic composition, in particular for correcting a skin color disorder or performing a permanent or long-lasting make-up that includes a base carrying a plurality of resorbable microneedles for delivering the composition. The device being having a length (Lₙ) of each microneedle (3) that is less than or equal to 50 mm, and the microneedles include a skin-coloring cosmetic composition.

### TECHNICAL FIELD

The present invention relates to a microneedle device comprising an array of microneedles which can be used to deliver a composition to skin, scalp, or lips to perform a cosmetic treatment modifying the color thereof.

### BACKGROUND ART

It is known to use liquid skin-coloring compositions, such as inks containing pigments, to be delivered to the uppermost layers of skin or to the eye corneum, for dermal-pigmentation or keratinous-pigmentation. These compositions can provide a lasting but not permanent correction to local skin color disorders.

The Stratum Corneum (SC) constitutes the main barrier of the epidermis to exogenous substances. Techniques aimed at removing the SC barrier, such as tape-stripping and suction, laser, or thermal ablation are impractical, while needle-free injections have so far failed to replace known needle-based delivery, which can be painful and non-attractive for the users.

The concept of using a microstructured device consisting of a plurality of microneedles to breach the stratum corneum barrier was first proposed in the 1970s. The production of solid microneedles arrays has been described in the art, for example in the applications WO 2009/040548, US 2015/0141910, and WO 2016/076442. Microneedles have an advantage of potentially penetrating the stratum corneum, without the discomfort of known needles, and can be self-administered.

There is a need for improving the delivery of cosmetic compositions into the skin, scalp, or lips, especially the precision of the amount of composition delivered and the control of the depth of injection into the skin at the targeted location.

### DISCLOSURE OF THE INVENTION

An object of the present invention is a device for delivering to the skin, scalp, or lips a skin-coloring cosmetic composition, in particular for correcting a skin color disorder or performing a permanent or long-lasting make-up, comprising a base carrying a plurality of resorbable microneedles for delivering the composition, the device being characterized in that a length of each microneedle is less than or equal to 3 mm, and in that the microneedles comprise a skin-coloring cosmetic composition.

The invention offers a long lasting solution for skin, scalp, or lips coloration and color disorders correction, by improving skin penetration of the composition.

The device according to the invention allows targeting and delivering compositions into skin layers, to correct or erase skin color disorders, such as achromia, alopecia, vitiligo, scars, for example from acne, depigmented spot-areas, over-pigmented areas and spots, or also to attenuate the dark circles under the eyes.

Thanks to the microneedles, the composition is delivered deeper inside skin, by-passing the SC layer, which is not possible with topical applications.

The device of the invention is convenient to use for cosmetic, non-therapeutic, treatments.

Microneedles

The microneedles used according to the invention are known in the art.

The microneedles may be hollow. By “hollow microneedles”, it has to be understood that the microneedles are not solid.

Hollow microneedles are disclosed in many publications such as in the articles “Microneedles for transdermal drug delivery”, Advanced Drug Delivery Reviews, Volume 56, Issue 5, 27 Mar. 2004, Pages 581-587, “Biodegradable polymer microneedles: Fabrication, mechanics and transdermal drug delivery”, Journal of Controlled Release, Volume 104, Issue 1, 5 May 2005, Pages 51-66, “Microfabricated needles for transdermal delivery of macromolecules and nanoparticles: Fabrication methods and transport studies, transport studies”, Devin V. McAllister et al., PNAS November 25, 2003. 100 (24) 13755-13760, “Sharp beveled tip hollow microneedle arrays fabricated by LIGA and 3D soft lithography with polyvinyl alcohol”, F Perennes et al., published 25 Jan. 2006, IOP Publishing LtdJournal of Micromechanics and Microengineering, Volume 16, Number 3, “Microneedle array for transdermal biological fluid extraction and in situ analysis”, E. V.Mukerjeeab et al., Sensors and Actuators A: Physical, Volume 114, Issues 2-3, 1 Sep. 2004, Pages 267-275, or “Hollow Microneedle Arrays for Intradermal Drug Delivery and DNA Electroporation”, Liêvin Daugimont et al., The Journal of Membrane Biology, July 2010, Volume 236, Issue 1, pp 117-125.

Hollow microneedles are also known from internet publications, such as the one at https://www.ncbi.nlm.nih.gov/pmc/articles/PMC4588187/, concerning Transdermal Delivery of Drugs with Microneedles—Potential and Challenges, Pharmaceutics 2015 Sep. 7(3): 90-105, published online 2015 Jun. 29, or https://www.ncbi.nlm.nih.gov/pubmed/17940907, Pharmaceutics 2015 Sep; 7(3): 90-105, published online 2015 Jun. 29, concerning Membrane-sealed hollow microneedles and related administration schemes for transdermal drug delivery, or https://www. 3m.com/3M/en_US/drug-delivery-systems-us/. . . /microneedle/concerning 3M Microneedle Drug Delivery Systems, or https://link.springer.com/article/10.1007/s00542-012-1663-1 concerning Optimizing hollow microneedles arrays aimed at transdermal drug delivery, Microsystem Technologies, January 2013, Volume 19, Issue 1, pp 1-8, or https://www.tyndall.ie/transdermal-drug-delivery.

Hollow microneedles are also known from the patent application EP 2 876 602, describing a method for converting an image of an array of nanostructures to a representation in a coordinate system.

Patent application US 2011/0213335 discloses a rapid, high-volume, intradermal infusion with minimal pain, achieved by applying an array of several hollow microneedles into the skin of a patient.

Patent application US 2013/0116523 describes a method for fabricating a hollow microneedle having a variable appearance. The method makes it possible to vary the length of the microneedle, the outer and inner diameters of the upper and lower parts thereof, the aspect ratio, the sharpness, and the structural bending rate thereof, in accordance with the desired purposes of use.

Patent application U.S. Pat. No. 6,503,231 discloses a microneedle device for transport of therapeutic and diagnostic materials and/or energy across tissue barriers. The microneedles are hollow and/or porous and permit drug delivery, or removal or sensing of body fluids, at clinically relevant rates across skin or other tissue barriers, without damage, pain, or irritation to the tissue.

The hollow microneedles may have at least one internal channel. Such a channel may be longitudinal, that is to say running along a longitudinal axis of the microneedles, from their free ends to the base of the device, or transversal, that is to say running along an axis extending obliquely or perpendicularly to the longitudinal axis of the microneedles. The microneedles may have a plurality of channels, longitudinal and/or transversal. The channels running through the microneedles may be continuous or discontinuous.

The microneedles may have a circular hollow internal cross-section. In a variant, the microneedles have hollow internal cross-section of other shapes, for example square, rectangular, or triangular.

The microneedles may be made in a porous material, preferably able to be loaded with said at least one composition aimed at generating an in-situ reaction into the skin, scalp, or lips. The microneedles may be made in a sintered material.

In variants, the microneedles are solid. By “solid”, it has to be understood that the microneedles are without an internal cavity, and are of uniformly close and coherent texture.

By “resorbable microneedles”, or “bioresorbable, or naturally-dissolving”, it has to be understood that the microneedles dissolve or degrade in vivo, for example after between at least 10 seconds and 24 hours, preferably in less than 8 hours. The microneedles may be bioabsorbable or biodegradable. The microneedles may dissolve or degrade thanks to an enzymatic reaction. In the case of resorbable microneedles, the microneedles are advantageously manufactured from a material that may dissolve, be absorbed or broken down by the body and thus do not require any mechanical removal.

The microneedles may be soluble in any body fluid or suitable composition.

In a variant, resorbable microneedles may be swellable into the skin, scalp or lips, that is to say being able to increase in volume when injected into the skin, scalp or lips.

In the case of resorbable microneedles, at least part of the material in which are made the microneedles may be used, once injected into the skin, scalp or lips, as a skin-coloring composition.

In a variant where the microneedles are hollow and resorbable, at least part of the material in which are made the microneedles is preferably used, once injected into the skin, scalp or lips, to enhance the skin-coloring by reacting with a composition delivered by the hollow microneedles.

A length of each microneedle is preferably less than or equal to 50 mm, better to 20 mm, better to 5 mm, better to 1 mm, better to 0.3 mm, even better to 0.1 mm. The length of a microneedle is measured, along its elongation axis, from its free end to the point where it connects to the base. The expression “elongation axis of a microneedle” denotes an axis which passes through the centers of mass of the cross sections of the microneedle.

An external largest transverse dimension of each microneedle, measured at the point where it is attached to the base, perpendicularly to its elongation axis, may be less than or equal to 1500 micrometers, better to 1000 micrometers, even better to 300 micrometers. The external cross-dimension of the microneedles advantageously decreases regularly towards their free ends.

The internal volume of each microneedle may be less than or equal to 10 mm³, better than 5 mm³, even better than 3 mm³.

The microneedles may be configured to deliver the composition at a flow rate less than or equal to 3 cm³/min, better to 0.3 cm³/min. Each microneedle may comprise a stop configured for limiting the depth of injection of the microneedle into the skin, scalp, or lips to less than or equal to 500 micrometers, better to 200 micrometers, even better to 100 micrometers.

An internal largest transverse dimension of each microneedle, i.e. its diameter when the microneedle has a circular hollow cross-section, may be less than or equal to 1000 micrometers, better to 500 micrometers, even better to 200 micrometers. The internal largest transverse dimension of the microneedles may be chosen according to the required volume of composition to be distributed.

The microneedles are preferably longer than the desired depth of injection. The length of the microneedles may be chosen according to the targeted skin layer into which the composition has to be delivered, obtaining the appropriated depth into the skin.

The microneedles may be oriented perpendicular to a substantially planar surface along which the base extends. In a variant embodiment, the microneedles are oriented obliquely to the substantially planar surface along which the base extends. This allows delivering the composition to curved surfaces with locally oriented perpendicularly microneedles.

The microneedles may have a curved shape. The microneedles may be of non-cylindrical shape, especially pyramidal with an octagonal base. The article of O'Mahony C., “Structural characterization and in-vivo reliability evaluation of silicon microneedles”, Biomed Microdevices, 2014, 16(3):333-43 shows that conical shape leads to very high reliability for silicon microneedles during skin penetration. In variants, the microneedles may be of conical shape with other polygonal bases such as a hexagonal base, or of square shape.

The microneedles may be made of an inorganic material, preferably silicon, titanium, stainless steel, cobalt, ceramics, polyethylene, or any material than can be skin and/or body implantable. The material used for the microneedles may include a preservative, especially in the case of ceramics.

In a variant, the microneedles are made of an organic material, preferably a polymer, for example a Gantrez polymer, or sugar, polysaccharide, polyethylene, cellulose, or hyaluronic acid.

The material used for the microneedles may be able to carry light and/or heat and/or cold.

The microneedles are preferably sterile or sterilized before use. The microneedles are preferably of single use.

Device, control, communication The base may be a flexible substrate, preferably coated with adhesive. The device may comprise a plurality of rigid bases each carrying one or more microneedles, preferably integral therewith, and a flexible support to which the bases are fixed so that the bases can move relative to the other to conform to skin profile.

The device may be made of several bases connected together to one or different containers containing a composition to be injected.

The containers may be removed from the device and replace by a new one while the microneedles are still into the skin.

The containers may be refillable while still connected to an array of microneedles.

The base may carry between 2 and 1000 microneedles per cm', better between 15 and 50 microneedles. The number of microneedles by base advantageously depends on the targeted area of the skin to be treated. The number of microneedles on a base is advantageously related to the size of the base, which depends on the size of the area of the skin to be treated.

The device may be able to make the microneedles puncturing the skin at a deep level, then to distribute the composition while removing the device. This allows having a good distribution of the composition in the different skin layers.

The microneedles may be disposed regularly on the base. In a variant, the microneedles are non-regularly disposed on the base.

The microneedles of a base may have different lengths, different volumes, different shapes, and may be made of different materials.

The device may be connected by wireless communication to an electronic system, in particular a personal computer or a smartphone, for injection control. In a variant, the device comprises an electronic system.

The device may be able to deliver heat, cold, ultrasounds, massage, microcurrents, or light, to create a synergic effect on composition performance or to enable chemical reactions of the different compositions. Moreover, heat can be useful to maintain a low viscosity of the composition to ease its injection. Cold may help to harden the skin and to help skin being punctured by the microneedles. This delivering may be controlled by the electronic system.

The targeted area of skin may be sucked up into a chamber to bring the targeted area into contact with the microneedles. Sucking the skin helps the microneedles puncturing the skin, and also having the same penetration depth of all the microneedles into the skin. To this end, it may be used a sucking device comprising:
a chamber with an opening configured to face the targeted area of the skin when the device is applied on the skin,
a surface within the chamber at a recessed position from the opening, and
a pressure source in communication with the chamber, at least for decreasing the pressure in the chamber and causing the targeted area to be sucked up in the chamber and to contact the surface, the surface comprising the microneedles according to the invention for puncturing the skin of the targeted area.

The device may be arranged for performing a color measurement of an area of skin, scalp, or lips, identifying therein a zone where the color of the skin, scalp, or lips is to be modified, determining a desired color correction, and determining an injection to perform into said zone to obtain the desired change of color based on optical properties of the composition, and the quantity injected through each microneedle and their relative localization in the area.

The electronic system may be arranged for comparing the color measurement of the zone to be modified to a beforehand performed color measurement of a healthy zone nearby to compute a difference value A.E. The desired color correction and the quantity to inject through each microneedle and their relative localization in the area may be determined as a function of said difference value A.E.

This allows adapting the treatment to the type of skin to be treated and to the type of skin color disorder, and obtaining great precision in correcting skin color disorders, avoiding proceeding to several deliveries.

Color measurements may be performed in the “Lab” color space, which describes mathematically all perceivable colors in three dimensions: L for lightness and a and b for the color opponents green-red and blue-yellow.

Color measurements may be performed by using a colorimeter, for example a Chroma Meter from Konica Minolta.

An application, in particular running on a smartphone, may be used for delivery control and user interface.

The device may be part of a watch connected to a smartphone, or to a distant device connected with a wire or wireless, for example by radio frequency, WIFI, or Bluetooth®.

The device may comprise a vibrating system for inducing vibrations into the microneedles. Such a vibrating system may allow the microneedles to puncture the skin, avoiding fluid blockage in the microneedles, enabling particles proper suspension and liquid diffusion into the skin. The vibrating system may further provide a massage to the treated area, allowing a better diffusion of the composition.

System and Compositions

Another object of the invention is a system comprising the device as defined above and at least one skin-coloring cosmetic composition, in particular for correcting a skin color disorder or performing a permanent or long-lasting make-up. The composition is advantageously a liquid cosmetic composition comprising solid particles, preferably at least one pigment, to be injected.

The composition is preferably a biocompatible cosmetic composition. By “biocompatible”, it has to be understood a composition capable of fulfilling a specific function with an appropriate response from the skin.

The composition is preferably sterile, and of single use.

The composition may comprise an aqueous phase and at least one pigment, one especially chosen from organic or mineral pigments, in particular non-lamellar mineral pigments.

This pigment is a pigment approved for use in cosmetics by CTFA and the FDA used in cosmetic formulations.

In a particular embodiment, said pigment may be water-dispersible.

In another embodiment, the pigment may be oil-dispersible or with limited solubility in water.

In variants, the pigment may be oil-soluble and/or water-soluble.

In some embodiments, this pigment is a non-lamellar mineral pigment, more preferably a metal oxide, such as iron oxides, titanium dioxide, aluminum oxide, zirconium oxides, zinc oxide, or composite oxides, particularly an iron oxide selected from red iron oxide, yellow iron oxide or black iron oxide, or a mixture thereof, or even Prussian blue, manganese violet, or ultramarine blue.

The compositions considered according to the invention may comprise in particular at least one compound selected from (and their mixtures):

| Color | Commercial Name | Chemical Name | Supplier Name |
|---|---|---|---|
| Black | Sunpuro Black Iron Oxide C33-7001 | Iron Oxide | Sun |
| Black | Unipure Triple Black Lc990 | Iron Oxide | Sensient |
| Black | Unipure Black Lc902 | Carbon Black | Sensient |
| Black | Wd-Cb2 | Carbon Black | Daito |
| Black | Black Nf | Iron Oxide | Kobo |
| Blue | Suncroma Iron Blue C38-5410 | Ferric Ferrocyanide | Sun |
| Blue | Unipure Blue Lc 621 | Blue 1 Lake | Sensient |
| Blue | Unipure Blue Lc 686 | Ultramarine Blue | Sensient |
| Blue | Unipure Blue Lc 520 | Carmine | Sensient |
| Bordeaux | Suncroma D & C Red 34 Ca Lake C | Red 34 Lake | Sun |
| | 24-012 | | |
| Green | Unipure Green Lc 788 | Chromium Oxide Green | Sensient |
| Orange | Suncroma Fd & C Yellow 6 Al Lake | Yellow 6 Lake | Sun |
| | C70-5270 | | |
| Pink | Suncroma D & C Red 28 Al Lake | Red 28 Lake | Sun |
| | C14-6623 | | |
| Pink | Dell Red C-14-6634/Suncroma | Red 22 Lake | Sun |
| | D & C Red 22 Al Lake C14-6634 | | |
| Pink | Unipure Pink Lc 589 | Ultramarines | Sensient |
| Red | Unipure Red Lc 3079 Or | Red 7 | Sensient |
| Red | Carmin Covalac W 3508/Cosmetic | Carmine | Sensient/Eusa |
| | Carmine 09350/Carmin Rmam 52% | | Colors |
| Red | Unipure Red Lc 320 | Carmine | Sensient/Eusa |
| | Carmihpc 54% | | Colors |
| Red | Suncroma D & C Red 6 Ba Lake C- | Red 6 Lake | Sun |
| | 19-012 | | |
| Red | Suncroma D & C Red 6 Na Salt C19- | Red 6 Na Salt | Sun |
| | 6619 | | |
| Red | Suncroma D & C Red 7 Ca Lake C- | Red 7 Lake | Sun |
| | 19-003 | | |
| Red | Suncroma D & C Red 7 Ca Lake C | Red 7 Lake | Sun |
| | 19-011 | | |
| Red | Suncroma D & C Red 7 Ca Lake C19- | Red 7 Lake | Sun |
| | 025 | | |
| Red | Suncroma D & C Red 30 al Lake C37- | Red 30 Lake | Sun |
| | 038 | | |
| Red | Suncroma D & C Red 33 Al Lake | Red 33 Lake | Sun |
| | C17-6444 | | |
| Red | Suncroma Fd & C Red 40 Al Lake | Red 40 Lake | Sun |
| | C37-6340 | | |
| Red | Sunpuro Red Iron Oxide C33-8001 | Iron Oxide | Sun |
| Red | Unipure Red Lc 383 | Iron Oxide | Sensient |
| Violet | Unipure Violet Lc 581 | Manganese Violet | Sensient |
| Violet | Unipure Violet Lc 587 | Ultramarine Violet | Sensient |
| Violet | Unipure Blue Lc 520 | Carmine | Sensient |
| White | Hombitan Anatase Ff Pharma | Titanium Dioxide | Sachtleben |
| White | HOMBITAN AFDC300 | TITANIUM DIOXIDE | SACHTLEBEN |
| | (Used For NAI-TAO) | | |
| White | Tipaque Pf-671 | Titanium Dioxide | Ishihara Sangyo |
| White | Tipaque Pfc407 | Titanium Dioxide | Ishihara Sangyo |
| White | Sachtleben Rc 402 | Titanium Dioxide | Hunstman |
| White | KRONOS 1171 | TITANIUM DIOXIDE | KRONOS |
| | (Used NAI TAO) | | |
| White | Tipaque Cr-50 | Titanium Dioxide | Ishihara Sangyo |
| Yellow | Suncroma Fd & C Yel 5 Al Lk C69- | Yellow 5 Lake | Sun |
| | 4424 | | |
| Yellow | Fd & C Yel 5 Al W001 | Yellow 5 Lake | Sensient |
| Yellow | Suncroma Fd & C Yel 5 Al Lk | Yellow 5 Lake | Sun |
| | C69002 | | |
| Yellow | Unipure Yellow Lc124 | Yellow 10 Lake | Sensient |
| Yellow | Sunpuro Yellow Iron Oxide C33- | Iron Oxide | Sun |
| | 9001 | | |
| Yellow | Suncroma Fd & C Yellow 6 Al Lake | Yellow 6 Lake | Sun |
| | C70-5270 | | |

The composition may also comprise at least one organic pigment, as for example a diketopynolopynoles (DPP), as the ones described in EP-A-542669, EP-A-787730, EP-A-787731 and WO-A-96/08537.

The composition is for example one of the solutions of bio-pigments BioChromaDerm or BioChromaEyes from Biotic Phocea.

The pigment may be melanin.

The largest dimension of the pigments may range between 0.1 micrometers and 300 micrometers, preferably between 0.2 micrometers and 100 micrometers. The dimension of the pigments may be adjusted to fit the internal channel of the microneedles.

The aqueous phase may account for at least 50% by weight relative to the weight of the composition.

The aqueous phase may comprise or consist in glycols, water, propane 1, 3, diol, or ethanol, and their mixtures.

The composition may be diluted before injection to the skin. In such a case, the minimum of oil and/or water before dilution may be 0%. In variants, the composition is a lipo-soluble or hydro-soluble coloring agent.

The concentration of such coloring agent in water or oil may be very low to generate skin color modification, preferably from 0.01% to 5% by weight.

In another variant, the composition is a liquid composition comprising high refraction index particles, interference particles, reflection particles, light absorbent particles, in particular non-transparent particles or particles modifying the path of light, in order to prevent or enhance reflection, or to reduce or hide light. The composition may be a liquid composition comprising coloring, bluffing, or matifiant particles. The particles may be particles of nacre, silica, starch, bentonite, or clay. Such a composition allows coloring or dermal-pigmenting the skin thanks to the optical properties of the particles they contain. The composition may be able to protect skin from ultra-violet damages or to bring skin glow.

In yet another variant, the composition may comprise hydroxyapatite particles or insoluble calcium alginate particles or aluminum oxide or noble metals, such as gold.

The depth of injection may be chosen according to the desired effect and may also depend on the desired duration of the effect, the length being greater for example when a long-lasting effect is required.

The viscosity of the composition may range from 1.10⁻³ Pa s⁻¹ to 10000 Pa s⁻¹, preferably from 1.10⁻³ Pa s⁻¹ to 3000 Pa s⁻¹.

The viscosity is measured at 25 ° C. and under 1 atm, with a Rheomat 180 viscometer equipped with MK-R-1, 2 or 3 mobile according to the viscosity range and the corresponding measurement cup MB-R-1, 2 or 3 at a rotation speed of 200 min⁻¹, the measurement being carried out after 10 minutes of rotation (time at which the stabilization of the viscosity and the speed of rotation of the mobile is observed).

The features defined above for the device apply to the system and vice-versa.

Flexible System

The device according to the invention may be embedded on a flexible and wearable system arranged for conforming to the area of the skin where the composition has to be delivered.

Such a wearable system may be made of a non-woven, absorbent material such as for example foam, latex, polyurethane, or film. Such a wearable system may be made of a resorbable material.

The thickness of the wearable system may lie between 5p,m to 3mm, preferably between 15 μm to 500 μm.

The wearable system may include a holding device to help its fixing on the treated area of the skin. The wearable system may include an adhesive polymer for fixing.

Kit

Yet another object of the invention is a kit comprising the microneedles device according to the invention and one or several skin-coloring cosmetic compositions, in particular for correcting a skin color disorder or performing a permanent or long-lasting make-up.

The compositions may be as defined above.

The features defined above for the device apply to the kit and vice-versa.

Method for Preparing an Injection of a Skin-Coloring Composition

Another object of the invention is a method for preparing an injection of a skin-coloring cosmetic composition, into an area of skin, scalp, or lips, the injection being performed with the device as defined above, the method comprising:

identifying a zone where the color of the skin, scalp, or lips is to be modified,

determining a desired color correction, and

determining an injection to perform into said zone to obtain the desired change of color based on optical properties of the composition, and the quantity to be injected through each microneedle and their relative localization in the area.

A color measurement of an area of skin, scalp, or lips comprising said zone may be performed. The color measurement of the zone to be treated may be compared to a beforehand performed color measurement of a healthy zone nearby to compute a difference value ΔE, and the desired color correction and the quantity to inject through each microneedle and their relative localization in the area may be determined as a function of said difference value ΔE.

The injected composition may be the same for each microneedle. In a variant, the injected composition is different for at least two microneedles of the device. The injected composition may be different for some zones of the area.

The position of the device on the skin, scalp, or lips may be automatically detected. The quantity of composition to be locally injected may be controlled as a function of the detected position and of the correction to be applied.

In a preferred embodiment, the microneedles are pre-filled with the composition before the application of the base on the skin. This allows avoiding the risk of injection of air.

In a variant, the microneedles are empty when the support is applied on the skin, and filled with the composition after the application on the skin.

Method of Cosmetic Treatment

Another object of the invention is a method of cosmetic treatment for modifying the color of an area of skin, comprising delivering into the area a skin-coloring cosmetic composition, prepared by the method as defined above.

The depth of injection of the microneedles into the skin is preferably less than or equal to 500 micrometers, better to 200 micrometers, even better to 100 micrometers. This range of depths corresponds to the area above the dermo-epidermal junction, and avoids bleeding and thus offers a comfortable use of the device.

The depth of injection into the skin advantageously depends on the composition: lighter, finer compositions can be associated with superficial injection whilst heavier, coarser compositions are preferably injected deeper.

A pre-solution may be applied beforehand on the skin to increase solubility kinetics of the microneedles and allow their removal, if needed, after delivery of the composition, especially a solution compatible for injection into the skin and/or topical application, for example a saline solution. Such a pre-solution may help to prepare skin but also to ease skin perforation. Such a pre-solution may contain active compounds and or polymers dedicated for skin surface treatment and or diffusion.

At least one control of skin dermal-pigmentation after use of a device according to the invention may be run, preferably several controls apart from a predefined period of time, for example one to four weeks.

The features defined above for the device apply to the methods and vice-versa.

### DESCRIPTION OF THE DRAWINGS

The foregoing aspects and many of the attendant advantages of the disclosed subject matter will become more readily appreciated as the same become better understood by reference to the following detailed description, when taken in conjunction with the accompanying drawings, wherein:

FIG. 1 shows a schematic embodiment of a microneedles device according to the invention; and

FIG. 2 is a block diagram illustrating some steps of a method for preparing an injection of a skin-coloring cosmetic composition into an area of skin, according to the invention.

### DETAILED DESCRIPTION

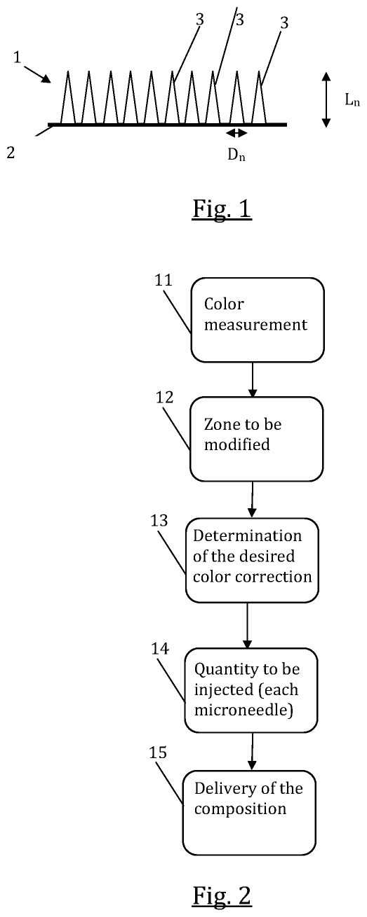

A device 1 according to the invention for delivering a skin-coloring cosmetic composition to the skin is shown in FIG. 1. In this example, such a device 1 comprises a base 2 carrying a plurality of hollow microneedles 3, each microneedle having an internal channel 3a running from their free ends to the base 2, presenting an internal volume for containing the composition to deliver to the skin up to 10 mm³.

The microneedles 3 are advantageously configured to deliver the composition at a flow rate less than or equal to 3 cm³/min.

The base 2 is for example a flexible substrate.

The length Lₙ of each microneedle 3 is less than or equal to 50 mm, being for example equal to 0.7 mm The microneedle length is advantageously adjusted to target beyond the SC barrier and/or epidermis.

The external largest transverse dimension D, of a microneedle 3, visible in FIG. 1, is for example less than or equal to 1500 micrometers.

Preferably, the microneedles 3 are of pyramidal shape with an octagonal base, and are oriented perpendicular to a substantially planar surface along which the base extends.

The microneedles 3 are made of an inorganic material, for example silicon, or of an organic material, for example a polymer.

The microneedles 3 are resorbable in the illustrated example.

The device according to the invention may comprise a vibrating system, not shown, for inducing vibrations into the microneedles 3. In a variant, a pre-solution is applied beforehand on the skin to weaken the microneedles 3 and allow their removal after delivery of the composition, especially a saline solution.

Preferably and as in the considered example, the composition comprises an aqueous phase and at least one pigment, especially chosen from organic or mineral pigments.

In a variant, the composition is a lipo-soluble or hydro-soluble coloring agent.

In another variant, as previously described, the composition is a liquid composition comprising high refraction index particles, interference particles, reflection particles, light absorbent particles, in particular non-transparent particles or particles modifying the path of light, in order to prevent reflection.

Some exemplary steps of a method, according to the invention, for preparing an injection of a skin-coloring cosmetic composition into an area of skin, scalp, or lips, using the microneedles device 1, are now going to be described in reference with FIG. 2.

As previously defined, in a step 11, a color measurement of an area of skin, scalp, or lips is performed. In this example, a colorimeter is used, for example a Chroma Meter from Konica Minolta.

A zone of the area where the color of the skin, scalp, or lips is to be modified is then identified in a step 12.

In a step 13, a desired color correction is determined, and an injection to perform into said zone to obtain the desired change of color based on optical properties of the composition, and the quantity injected through each microneedle and their relative localization in the area are determined in a step 14. The color measurement of the zone to be modified is preferably compared to a beforehand performed color measurement of a healthy zone nearby to obtain a difference value ΔE. The desired color correction and the quantity to inject through each microneedle and their relative localization in the area are then determined as a function of said difference value ΔE.

The composition is then delivered into the area for modifying its color, in a step 15. In this example, the microneedles 3 are pre-filled with the composition before application of the base 2 on the area of the skin. The depth of injection of the microneedles 3 into the skin is preferably less than or equal to 500 micrometers.

As previously defined, the device 1 may be connected by wireless communication to an electronic system, not shown, in particular a personal computer or a smartphone, for injection control, or comprises an electronic system. Such electronic system is in particular arranged for controlling the injection of a predefined quantity of composition into the microneedles 3, by performing at least some of the previously-described method steps. An application, in particular running on a smartphone, may be used for delivery control and user interface.

### EXAMPLE 1

In a first example, a liquid skin color-changing composition is delivered, by using a device 1 according to the invention, on dark circles under the eyes. The composition is an under-eye corrector, comprising pigments, for example the composition Biochromaeyes® from BIOTEC PHOCEA.

The dark circles are covered by the composition in a satisfying manner.

### EXAMPLE 2

In a second example, a liquid skin color-changing composition is delivered, by using a device 1 according to the invention, on areas of the face affected by vitiligo. The composition comprises, in this example, a suspension of different pigments and/or coloring particles to match the proper targeted skin color, with the objective to treat the vitiligo sites to obtain the same color as the non-vitiligo sites. The composition may be the composition “Rosy corrector”, in the shade chocolate brown, from the Biochromaderm® compositions from BIOTEC PHOCEA.

The results are satisfactory, the appearance of areas around the mouth for example is enhanced and the effects of the treatment are quickly visible.

The container may comprise several rooms; each room being able to contain a different composition. The different rooms may be connected together. The container may be a syringe.

While illustrative embodiments have been illustrated and described, it will be appreciated that various changes can be made therein without departing from the spirit and scope of the claimed subject matter.

1. A device for delivering to the skin, scalp, or lips a skin-coloring cosmetic composition for correcting a skin color disorder or performing a permanent or long-lasting make-up, comprising a base carrying a plurality of resorbable microneedles for delivering the composition, the device a length of each microneedle that is less than or equal to 50 mm, and the microneedles comprise a skin-coloring cosmetic composition.

2. The device of claim 1, wherein the microneedles are hollow.

3. The device of claim 1, wherein the microneedles are solid.

4. The device of claim 1, wherein the microneedles are made in a porous material.

5. The device of claim 1, wherein each microneedle has an internal volume for containing the composition to deliver to the skin less than or equal to 10 mm³.

6. The device of claim 1, wherein the microneedles are configured to deliver the composition at a flow rate less than or equal to 3 cm³/min.

7. The device of claim 1, wherein a length of each microneedle is less than or equal to 20 mm.

8. The device of claim 1, wherein each microneedle comprises a stop configured for limiting the depth of injection of the microneedle into the skin to less than or equal to 500 micrometers.

9. The device of claim 1, wherein an external largest transverse dimension of each microneedle is less than or equal to 1500 micrometers.

10. The device of claim 1, wherein an internal largest transverse dimension of each microneedle is less than or equal to 1000 micrometers.

11. A system comprising the device according to claim 1 and at least one skin-coloring cosmetic composition to be injected for correcting a skin color disorder or performing a permanent or long-lasting make-up.

12. The system of claim 11, wherein the at least one skin-coloring cosmtic composition comprises solid particles.

13. The system of claim 11, wherein the at least one skin-coloring cosmetic composition comprises an aqueous phase and at least one pigment chosen from organic or mineral pigments.

14. The system of claim 11, wherein the at least one skin-coloring cosmetic composition is a lipo-soluble or hydro-soluble coloring agent.

15. The system of claim 11, wherein the at least one skin-coloring cosmetic composition is a liquid composition comprising high refraction index particles, interference particles, reflection particles, light absorbent particles, non-transparent particles or particles modifying the path of light, in order to revent reflection.

16. A kit comprising a device as defined in claim 1 and one or several skin-coloring cosmetic compositions for correcting a skin color disorder or performing a permanent or long-lasting make-up.

17. A method for preparing an injection of a skin-coloring cosmetic composition, into an area of skin, scalp, or lips, the injection being performed with the device as defined in claim 1, the method comprising:
identifying a zone where the color of the skin, scalp, or lips is to be modified,
determining a desired color correction, and
determining an injection to perform into said zone to obtain the desired change of color based on optical properties of the composition, and determining the quantity to be injected through each microneedle and relative localization in the area.

18. The method of claim 17, wherein a color measurement of an area of skin, scalp, or lips comprising said zone is performed, the color measurement of the zone to be treated being compared to a beforehand performed color measurement of a healthy zone nearby to compute a difference value, and a desired color correction and the quantity to inject through each microneedle and their relative localization in the area being determined as a function of said difference value.

19. The method of claim 17, wherein the injected composition is the same for each microneedle.

20. The method of claim 17, wherein the injected composition is different for at least two microneedles of the device and/or for some zones of the area.
